(19) United States
(12) Patent Application Publication
(10) Pub. No.: US 2023/0273148 A1
(43) Pub. Date: Aug. 31, 2023

(54) NOx SENSOR AND NOx SENSOR OPERATION METHOD

(71) Applicant: NGK INSULATORS, LTD., Nagoya-shi (JP)

(72) Inventors: Daichi ICHIKAWA, Nagoya-shi (JP); Yusuke WATANABE, Nagoya-shi (JP)

(21) Appl. No.: 18/171,908

(22) Filed: Feb. 21, 2023

(30) Foreign Application Priority Data
Feb. 25, 2022 (JP) 2022-027959
Feb. 7, 2023 (JP) 2023-016732

(51) Int. Cl. G01N 27/406; G01N 33/00; G01N 27/41

(52) U.S. Cl. CPC G01N 27/4065; G01N 27/41; G01N 33/0027

(57) ABSTRACT

A controller of a NOx sensor determines whether a determination target value being an indicator of operation of pumping in oxygen of one of electrochemical pump cells other than a measurement pump cell including inner electrodes arranged in respective internal spaces of a sensor element, an out-of-space pump outer electrode disposed at a location other than the internal spaces, and solid electrolytes between these electrodes exceeds a threshold during a predetermined determination time, controls, unless the determination target value exceeds the threshold, the NOx sensor in a basic mode in which a concentration of NOx is identified based on a pump current flowing between the measurement electrode and the outer electrode, and stops, when the determination target value exceeds the threshold, the basic mode, and controls the NOx sensor in a protected execution mode in which the measurement electrode is protected against over-reduction.

### CROSS-REFERENCE TO RELATED APPLICATION

The present application claims priority from Japanese applications JP2022-027959 filed on Feb. 25, 2022, and JP2023-016732 filed Feb. 7, 2023, the contents of which are hereby incorporated by reference into this application.

### BACKGROUND OF THE INVENTION

### Field of the Invention

The present invention relates to a limiting current type NOx sensor, and, in particular, to control of operation of the NOx sensor in use under a rich atmosphere.

### Description of the Background Art

A limiting current type NOx sensor including a sensor element containing an oxygen-ion conductive solid electrolyte, such as yttria stabilized zirconia, as a main component has already been known, for example. In the NOx sensor, a measurement gas is introduced sequentially into a plurality of spaces (internal spaces) arranged continuously inside the sensor element. Control to maintain a potential difference between each of a plurality of inner electrodes arranged to face the respective internal spaces and a reference electrode disposed inside the element and to be in contact with a reference gas at a predetermined value in accordance with a desired oxygen concentration in a space is performed.

The control is generally performed by applying, in electrochemical pump cells each including one of the plurality of inner electrodes, an outer (out-of-space) electrode disposed outside the space, and a solid electrolyte region present between the electrodes, a pumping voltage across the electrodes to pump in or out oxygen between the internal space and an outside. Due to application of the pumping voltage, an oxygen pumping current having a magnitude and a direction in accordance with an oxygen concentration in the space flows between the inner electrode and the outer electrode.

In particular, a potential difference between a measurement electrode as an inner electrode disposed in the farthest internal space and the reference electrode is controlled at a value at which all of oxygen generated by reduction of NOx is pumped out. The NOx sensor identifies a concentration of NOx based on a magnitude of the oxygen pumping current flowing between the measurement electrode and the outer electrode during control.

As one example of such a gas sensor, a gas sensor in which an outer electrode is disposed on an outer surface of a sensor element, and a ceramic layer is disposed so that a slit for providing predetermined diffusion resistance is formed around the outer electrode has already been known (see Japanese Patent Application Laid-Open No. 2021-162465, for example).

A gas sensor including a sensor element having a configuration in which an oxygen concentration detection cell and an oxygen pump cell are laminated via an insulating layer along a thickness direction of the element and a detection gas is introduced into the element through a diffusion control part formed of a porous body provided to a portion of the insulating layer has also already been known (see Japanese Patent Application Laid-Open No. 2012-173146, for example).

A limiting current type NOx sensor as described above is sometimes used in an environment in which a rich gas having an air-fuel ratio smaller than a theoretical air-fuel ratio can be introduced into the element, for example, along an exhaust path from a gasoline engine.

In this case, when the rich gas is introduced into an internal space, operation (pumping operation) of pumping in oxygen from outside the element to the internal space is typically performed in an electrochemical pump cell to maintain an oxygen concentration in the space constant. That is to say, a pumping voltage is applied so that oxygen is pumped in to the internal space (oxygen ions move from outside the element to the internal space), and an oxygen pumping current in accordance with the pumping voltage flows between an inner electrode and an outer electrode.

In pumping in oxygen, the pumping voltage and the oxygen pumping current tend to increase with increasing amount of the rich gas introduced into the internal space. An excessive increase in richness of the measurement gas, however, makes it difficult to pump in oxygen from an outside in accordance with the increase in pumping voltage, and, a measurement electrode, which is disposed in the farthest portion of a distribution part for the measurement gas in many cases, is over-reduced by the rich gas. Over-reduction of the measurement electrode occurring once causes a problem in that, even after the measurement gas introduced into the internal space has the theoretical air-fuel ratio or becomes lean, it takes time (e.g., approximately 20 minutes) to stabilize the measurement electrode, and a NOx concentration cannot accurately be measured during the time. Stabilization of the measurement electrode can be determined based on stabilization of the oxygen pumping current flowing between the measurement electrode and the outer electrode.

### SUMMARY

The present invention relates to a limiting current type NOx sensor, and, in particular, is directed to control of operation of the NOx sensor in use under a rich atmosphere.

According to the present invention, a NOx sensor capable of sensing NOx in a measurement gas includes: a sensor element formed of an oxygen-ion conductive solid electrolyte; and a controller controlling operation of the NOx sensor, wherein the sensor element includes: a plurality of internal spaces which communicate sequentially from an inlet for the measurement gas under predetermined diffusion resistance, and in which inner electrodes are respectively arranged; an out-of-space pump electrode disposed at a location other than the plurality of internal spaces; a reference electrode disposed to be contactable with a reference gas; a plurality of electrochemical pump cells configured to pump in or out oxygen between the plurality of internal spaces and an outside of the sensor element by applying pump voltages across the respective inner electrodes and the out-of-space pump electrode from predetermined pump power supplies; and a plurality of electrochemical sensor cells configured to cause potential differences between the respective inner electrodes and the reference electrode in accordance with oxygen concentrations in the respective internal spaces, one of the inner electrodes arranged in the respective internal spaces is a measurement electrode capable of reducing NOx, the plurality of electrochemical pump cells include: a measurement pump cell including the measurement electrode; and at least one oxygen concentration control pump cell including an inner electrode other than the measurement electrode, the plurality of electrochemical sensor cells include: a measurement sensor cell including the measurement electrode; and at least one oxygen concentration sensing sensor cell including the inner electrode other than the measurement electrode, and the controller determines whether a predetermined determination target value exceeds a predetermined threshold during a predetermined determination time, the determination target value being an indicator of operation of pumping in oxygen of a determination target pump cell included in the at least one oxygen concentration control pump cell, controls, unless the determination target value exceeds the predetermined threshold, the NOx sensor in a basic mode in which a concentration of NOx is identified based on a measurement pump current flowing between the measurement electrode and the out-of-space pump electrode of the measurement pump cell in accordance with the concentration of NOx due to reduction of NOx by the measurement electrode while an oxygen concentration in each of the plurality of internal spaces is maintained constant by operating each of the plurality of electrochemical pump cells, and stops, when the determination target value exceeds the predetermined threshold, the basic mode, and controls the NOx sensor in a protected execution mode in which the measurement electrode is protected against over-reduction.

According to the present invention, an oxygen concentration uncontrollable condition due to difficulty in pumping in oxygen to the internal spaces is suitably avoided even when the NOx sensor is used in an environment in which the measurement gas might be a rich gas having a small air-fuel ratio. In addition, over-reduction of the measurement electrode by the rich gas is suitably suppressed, so that use of the measurement electrode can promptly be enabled in resuming measurement of NOx.

It is thus an object of the present invention to provide a NOx sensor capable of protecting a measurement electrode in use under a rich atmosphere.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a diagram schematically showing one example of a configuration of a gas sensor 100;

FIG. 2 is a diagram showing an operational flow in a first aspect of an element protection mode;

FIG. 3 is a diagram showing an operational flow in a second aspect of the element protection mode;

FIG. 4 is a diagram showing an operational flow in a third aspect of the element protection mode;

FIG. 5 is a diagram showing an operational flow in the third aspect of the element protection mode; and

FIG. 6 is a diagram schematically showing one example of a configuration of a gas sensor 100B.

### DESCRIPTION OF THE PREFERRED EMBODIMENTS

### General Configuration of Gas Sensor

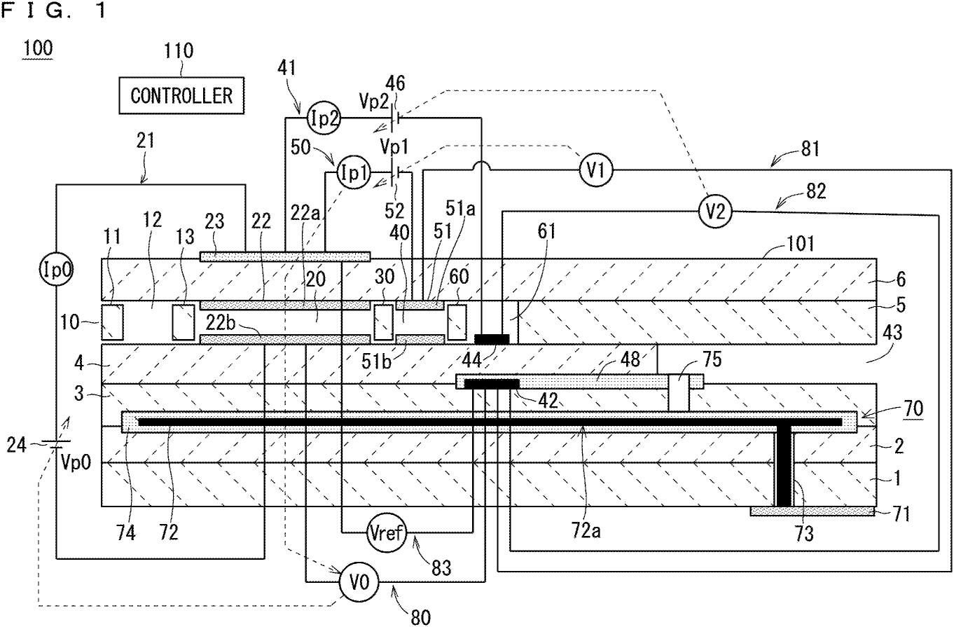

FIG. 1 is a diagram schematically showing one example of a configuration of a gas sensor 100 according to the present embodiment. The gas sensor 100 is a limiting current type NOx sensor sensing NOx and measuring a concentration thereof using a sensor element 101. The gas sensor 100 further includes a controller 110 controlling operation of each part and identifying the NOx concentration based on a NOx current flowing through the sensor element 101. FIG. 1 includes a vertical cross-sectional view taken along a longitudinal direction of the sensor element 101.

The sensor element 101 is a planar (an elongated planar) element body of ceramics having a structure in which six solid electrolyte layers, namely, a first substrate layer 1, a second substrate layer 2, a third substrate layer 3, a first solid electrolyte layer 4, a spacer layer 5, and a second solid electrolyte layer 6 each formed of zirconia (ZrO₂) (e.g., yttria stabilized zirconia (YSZ)) as an oxygen-ion conductive solid electrolyte are laminated in the stated order from a bottom side of FIG. 1. The solid electrolyte forming these six layers is dense and airtight. A surface on an upper side and a surface on a lower side of each of these six layers in FIG. 1 are hereinafter also simply referred to as an upper surface and a lower surface, respectively. A part of the sensor element 101 formed of the solid electrolyte as a whole is generically referred to as a base part.

The sensor element 101 is manufactured, for example, by performing predetermined processing, printing of circuit patterns, and the like on ceramic green sheets corresponding to the respective layers, then laminating them, and further firing them for integration.

Between a lower surface of the second solid electrolyte layer 6 and an upper surface of the first solid electrolyte layer 4 in one leading end portion of the sensor element 101, a first diffusion control part 11 doubling as a gas inlet 10, a buffer space 12, a second diffusion control part 13, a first internal space 20, a third diffusion control part 30, a second internal space 40, a fourth diffusion control part 60, and a third internal space 61 are formed adjacent to each other to communicate in the stated order.

The buffer space 12, the first internal space 20, the second internal space 40, and the third internal space 61 are spaces (regions) inside the sensor element 101 looking as if they were provided by hollowing out the spacer layer 5, and having an upper portion, a lower portion, and a side portion respectively defined by the lower surface of the second solid electrolyte layer 6, the upper surface of the first solid electrolyte layer 4, and a side surface of the spacer layer 5. The gas inlet 10 may similarly look as if it was provided by hollowing out the spacer layer 5 at a leading end surface (at the left end in FIG. 1) of the sensor element 101 separately from the first diffusion control part 11. In this case, the first diffusion control part 11 is formed inside and adjacent to the gas inlet 10.

The first diffusion control part 11, the second diffusion control part 13, the third diffusion control part 30, and the fourth diffusion control part 60 are each provided as two horizontally long slits (whose openings have longitudinal directions perpendicular to the page of FIG. 1). A part extending from the gas inlet 10 to the third internal space 61 is also referred to as a gas distribution part.

At a location farther from the leading end than the gas distribution part is, a reference gas introduction space 43 having a side portion defined by a side surface of the first solid electrolyte layer 4 is provided between an upper surface of the third substrate layer 3 and a lower surface of the spacer layer 5. For example, air is introduced into the reference gas introduction space 43 as a reference gas at measurement of the NOx concentration.

An air introduction layer 48 is a layer formed of porous alumina, and the reference gas is introduced into the air introduction layer 48 through the reference gas introduction space 43. The air introduction layer 48 is formed to cover a reference electrode 42.

The reference electrode 42 is an electrode formed to be sandwiched between the upper surface of the third substrate layer 3 and the first solid electrolyte layer 4, and the air introduction layer 48 leading to the reference gas introduction space 43 is provided around the reference electrode 42 as described above. As will be described below, an oxygen concentration (oxygen partial pressure) in the first internal space 20 and the second internal space 40 can be measured using the reference electrode 42.

In the gas distribution part, the gas inlet 10 (first diffusion control part 11) is a part opening to an external space, and a measurement gas is taken from the external space into the sensor element 101 through the gas inlet 10.

The first diffusion control part 11 is a part providing predetermined diffusion resistance to the taken measurement gas.

The buffer space 12 is a space provided to guide the measurement gas introduced through the first diffusion control part 11 to the second diffusion control part 13.

The second diffusion control part 13 is a part providing predetermined diffusion resistance to the measurement gas introduced from the buffer space 12 into the first internal space 20.

In introducing the measurement gas from outside the sensor element 101 into the first internal space 20, the measurement gas having abruptly been taken into the sensor element 101 through the gas inlet 10 due to pressure fluctuations (pulsation of exhaust pressure in a case where the measurement gas is an exhaust gas of a vehicle) of the measurement gas in the external space is not directly introduced into the first internal space 20 but is introduced into the first internal space 20 after concentration fluctuations of the measurement gas are canceled through the first diffusion control part 11, the buffer space 12, and the second diffusion control part 13. This makes the concentration fluctuations of the measurement gas introduced into the first internal space 20 almost negligible.

The first internal space 20 is provided as a space to adjust oxygen partial pressure of the measurement gas introduced through the second diffusion control part 13. The oxygen partial pressure is adjusted by operation of a main pump cell 21.

The main pump cell 21 is an electrochemical pump cell including an inner pump electrode 22, an outer (out-of-space) pump electrode 23, and the second solid electrolyte layer 6 sandwiched between these electrodes. The inner pump electrode 22 has a ceiling electrode portion 22a provided on substantially the entire lower surface of a portion of the second solid electrolyte layer 6 facing the first internal space 20, and the outer pump electrode 23 is provided in a region, on an upper surface of the second solid electrolyte layer 6 (one main surface of the sensor element 101), corresponding to the ceiling electrode portion 22a to be exposed to the external space.

The inner pump electrode 22 is formed on upper and lower solid electrolyte layers (the second solid electrolyte layer 6 and the first solid electrolyte layer 4) defining the first internal space 20. Specifically, the ceiling electrode portion 22a is formed on the lower surface of the second solid electrolyte layer 6, which provides a ceiling surface to the first internal space 20, and a bottom electrode portion 22b is formed on the upper surface of the first solid electrolyte layer 4, which provides a bottom surface to the first internal space 20. The ceiling electrode portion 22a and the bottom electrode portion 22b are connected by a conducting portion (not illustrated) provided on a side wall surface (an inner surface) of the spacer layer 5 forming opposite side wall portions of the first internal space 20.

The ceiling electrode portion 22a and the bottom electrode portion 22b are provided to be rectangular in plan view. Only the ceiling electrode portion 22a or only the bottom electrode portion 22b may be provided.

The inner pump electrode 22 and the outer pump electrode 23 are each formed as a porous cermet electrode. In particular, the inner pump electrode 22 to be in contact with the measurement gas is formed using a material having a weakened reducing ability with respect to a NOx component in the measurement gas. For example, the inner pump electrode 22 is formed as a cermet electrode of an Au-Pt alloy containing Au of approximately 0.6 wt% to 1.4 wt% and ZrO₂ to have a porosity of 5% to 40% and a thickness of 5 µm to 20 µm. A weight ratio Pt:ZrO₂ of the Au-Pt alloy and ZrO₂ is only required to be approximately 7.0:3.0 to 5.0:5.0.

On the other hand, the outer pump electrode 23 is formed, for example, as a cermet electrode of Pt or an alloy thereof and ZrO₂ to be rectangular in plan view.

The main pump cell 21 can pump out oxygen in the first internal space 20 to the external space or pump in oxygen in the external space to the first internal space 20 by applying a desired pump voltage Vp0 across the inner pump electrode 22 and the outer pump electrode 23 from a variable power supply 24 to allow a main pump current Ip0 to flow between the inner pump electrode 22 and the outer pump electrode 23 in a positive or negative direction. The pump voltage Vp0 applied across the inner pump electrode 22 and the outer pump electrode 23 of the main pump cell 21 is also referred to as a main pump voltage Vp0.

To detect the oxygen concentration (oxygen partial pressure) in an atmosphere in the first internal space 20, the inner pump electrode 22, the second solid electrolyte layer 6, the spacer layer 5, the first solid electrolyte layer 4, the third substrate layer 3, and the reference electrode 42 constitute a main sensor cell 80 as an electrochemical sensor cell.

The oxygen concentration (oxygen partial pressure) in the first internal space 20 can be known by measuring electromotive force V0 as a potential difference between the inner pump electrode 22 and the reference electrode 42 in the main sensor cell 80.

Furthermore, the controller 110 performs feedback control of the main pump voltage Vp0 so that the electromotive force V0 is constant, thereby to control the main pump current Ip0. The oxygen concentration in the first internal space 20 is thereby maintained at a predetermined constant value.

The third diffusion control part 30 is a part providing predetermined diffusion resistance to the measurement gas having an oxygen concentration (oxygen partial pressure) controlled by operation of the main pump cell 21 in the first internal space 20, and guiding the measurement gas to the second internal space 40.

The second internal space 40 is provided as a space to further adjust the oxygen partial pressure of the measurement gas introduced through the third diffusion control part 30. The oxygen partial pressure is adjusted by operation of an auxiliary pump cell 50. The oxygen concentration of the measurement gas is adjusted with higher accuracy in the second internal space 40.

After the oxygen concentration (oxygen partial pressure) is adjusted in advance in the first internal space 20, the auxiliary pump cell 50 further adjusts the oxygen partial pressure of the measurement gas introduced through the third diffusion control part 30 in the second internal space 40.

The auxiliary pump cell 50 is an auxiliary electrochemical pump cell including an auxiliary pump electrode 51, the outer pump electrode 23 (not limited to the outer pump electrode 23 and only required to be any appropriate electrode outside the sensor element 101), and the second solid electrolyte layer 6. The auxiliary pump electrode 51 has a ceiling electrode portion 51a provided on substantially the entire lower surface of a portion of the second solid electrolyte layer 6 facing the second internal space 40.

The auxiliary pump electrode 51 is provided in the second internal space 40 in a similar form to the inner pump electrode 22 provided in the first internal space 20 described previously. That is to say, the ceiling electrode portion 51a is formed on the second solid electrolyte layer 6, which provides a ceiling surface to the second internal space 40, and a bottom electrode portion 51b is formed on the first solid electrolyte layer 4, which provides a bottom surface to the second internal space 40. The ceiling electrode portion 51a and the bottom electrode portion 51b are rectangular in plan view, and are connected by a conducting portion (not illustrated) provided on the side wall surface (inner surface) of the spacer layer 5 forming opposite side wall portions of the second internal space 40.

As with the inner pump electrode 22, the auxiliary pump electrode 51 is formed using a material having a weakened reducing ability with respect to the NOx component in the measurement gas.

The auxiliary pump cell 50 can pump out oxygen in an atmosphere in the second internal space 40 to the external space or pump in oxygen in the external space to the second internal space 40 by applying a desired voltage (an auxiliary pump voltage) Vp1 across the auxiliary pump electrode 51 and the outer pump electrode 23 under control performed by the controller 110.

To control the oxygen partial pressure in the atmosphere in the second internal space 40, the auxiliary pump electrode 51, the reference electrode 42, the second solid electrolyte layer 6, the spacer layer 5, the first solid electrolyte layer 4, and the third substrate layer 3 constitute an auxiliary sensor cell 81 as an electrochemical sensor cell. In the auxiliary sensor cell 81, electromotive force V1 as a potential difference caused between the auxiliary pump electrode 51 and the reference electrode 42 in accordance with the oxygen partial pressure in the second internal space 40 is detected.

The auxiliary pump cell 50 performs pumping using a variable power supply 52 whose voltage is controlled based on the electromotive force V1 detected in the auxiliary sensor cell 81. The oxygen partial pressure in the atmosphere in the second internal space 40 is thereby feedback controlled to a low partial pressure having substantially no effect on measurement of NOx.

At the same time, a resulting auxiliary pump current Ip1 is used to control the electromotive force in the main sensor cell 80. Specifically, the auxiliary pump current Ip1 is input, as a control signal, into the main sensor cell 80, and, through control of the electromotive force V0 therein, the oxygen partial pressure of the measurement gas introduced through the third diffusion control part 30 into the second internal space 40 is controlled to have a gradient that is always constant. In use as the NOx sensor, the oxygen concentration in the second internal space 40 is maintained at a constant value of approximately 0.001 ppm by the action of the main pump cell 21 and the auxiliary pump cell 50.

The fourth diffusion control part 60 is a part providing predetermined diffusion resistance to the measurement gas having an oxygen concentration (oxygen partial pressure) controlled by operation of the auxiliary pump cell 50 in the second internal space 40, and guiding the measurement gas to the third internal space 61.

The third internal space 61 is provided as a space (measurement internal space) to perform processing concerning measurement of the nitrogen oxide (NOx) concentration of the measurement gas introduced through the fourth diffusion control part 60. The NOx concentration is measured by operation of a measurement pump cell 41 in the third internal space 61. The measurement gas having the oxygen concentration adjusted with high accuracy in the second internal space 40 is introduced into the third internal space 61, so that the NOx concentration can be measured with high accuracy in the gas sensor 100.

The measurement pump cell 41 is to measure the NOx concentration of the measurement gas introduced into the third internal space 61. The measurement pump cell 41 is an electrochemical pump cell including a measurement electrode 44, the outer pump electrode 23, the second solid electrolyte layer 6, the spacer layer 5, and the first solid electrolyte layer 4. The measurement electrode 44 is provided on an upper surface of a portion of the first solid electrolyte layer 4 facing the third internal space 61 to be separated from the third diffusion control part 30.

The measurement electrode 44 is a porous cermet electrode of a noble metal and a solid electrolyte. For example, the measurement electrode 44 is formed as a cermet electrode of Pt or an alloy of Pt and another noble metal, such as Rh, and ZrO₂ as a constituent material for the sensor element 101. The measurement electrode 44 also functions as a NOx reduction catalyst to reduce NOx present in an atmosphere in the third internal space 61.

The measurement pump cell 41 can pump out oxygen generated through decomposition of NOx in the atmosphere in the third internal space 61, and detect the amount of generated oxygen as a pump current Ip2 under control performed by the controller 110.

To detect the oxygen partial pressure around the measurement electrode 44, the second solid electrolyte layer 6, the spacer layer 5, the first solid electrolyte layer 4, the third substrate layer 3, the measurement electrode 44, and the reference electrode 42 constitute a measurement sensor cell 82 as an electrochemical sensor cell. A variable power supply 46 is feedback controlled based on electromotive force V2 as a potential difference caused between the measurement electrode 44 and the reference electrode 42 detected by the measurement sensor cell 82 in accordance with the oxygen partial pressure in the third internal space 61.

NOx in the measurement gas introduced into the third internal space 61 is reduced by the measurement electrode 44 (2NO → N₂ + O₂) to generate oxygen. Oxygen as generated is to be pumped by the measurement pump cell 41, and, in this case, a voltage (measurement pump voltage) Vp2 of the variable power supply 46 is controlled so that the electromotive force V2 detected by the measurement sensor cell 82 is constant. The amount of oxygen generated around the measurement electrode 44 is proportional to the NOx concentration of the measurement gas, and thus the NOx concentration of the measurement gas is to be calculated using the pump current Ip2 in the measurement pump cell 41. The pump current Ip2 is hereinafter also referred to as a NOx current Ip2.

In the case that the measurement electrode 44, the first solid electrolyte layer 4, the third substrate layer 3, and the reference electrode 42 are combined to constitute an oxygen partial pressure detection means as an electrochemical sensor cell, electromotive force in accordance with a difference between the amount of oxygen generated through reduction of a NOx component in the atmosphere around the measurement electrode 44 and the amount of oxygen contained in reference air can be detected, and the concentration of the NOx component in the measurement gas can thereby be determined.

The second solid electrolyte layer 6, the spacer layer 5, the first solid electrolyte layer 4, the third substrate layer 3, the outer pump electrode 23, and the reference electrode 42 constitute an electrochemical sensor cell 83, and oxygen partial pressure of the measurement gas outside the sensor can be detected using electromotive force Vref determined by the sensor cell 83.

The sensor element 101 further includes a heater part 70 playing a role in temperature adjustment of heating the sensor element 101 and maintaining the temperature thereof to enhance oxygen ion conductivity of the solid electrolyte forming the base part.

The heater part 70 mainly includes a heater electrode 71, a heater element 72, a heater lead 72a, a through hole 73, a heater insulating layer 74, a pressure dissipation hole 75, and a heater resistance detection lead, which is not illustrated in FIG. 1. A portion of the heater part 70 other than the heater electrode 71 is buried in the base part of the sensor element 101.

The heater electrode 71 is an electrode formed to be in contact with a lower surface of the first substrate layer 1 (the other main surface of the sensor element 101).

The heater element 72 is a resistive heating element provided between the second substrate layer 2 and the third substrate layer 3. The heater element 72 generates heat by being powered from a heater power supply, which is not illustrated in FIG. 1, outside the sensor element 101 through the heater electrode 71, the through hole 73, and the heater lead 72a, which constitute a current-carrying path. The heater element 72 is formed of Pt, or contains Pt as a main component. The heater element 72 is buried, in a predetermined range of the sensor element 101 in which the gas distribution part is provided, to oppose the gas distribution part in a thickness direction of the element. The heater element 72 is provided to have a thickness of approximately 10 µm to 20 µm.

In the sensor element 101, each part of the sensor element 101 can be heated to a predetermined temperature and the temperature can be maintained by allowing a current to flow through the heater electrode 71 to the heater element 72 to thereby cause the heater element 72 to generate heat. Specifically, the sensor element 101 is heated so that the temperature of the solid electrolyte and the electrodes in the vicinity of the gas distribution part is approximately 700° C. to 900° C. The oxygen ion conductivity of the solid electrolyte forming the base part of the sensor element 101 is enhanced by the heating. A heating temperature of the heater element 72 when the gas sensor 100 is in use (when the sensor element 101 is driven) is referred to as a sensor element driving temperature.

A degree of heat generation (heater temperature) of the heater element 72 is grasped by the magnitude of a resistance value (heater resistance) of the heater element 72.

Although not illustrated in FIG. 1, an electrode protective layer covering the outer pump electrode 23 may be provided on a side of the one main surface of the sensor element 101 to protect the outer pump electrode 23.

A thermal shock resistant protective layer as a single- or multi- porous layer covering the sensor element 101 may further be provided outside in a predetermined range on a side of the one leading end portion (side of the left end in FIG. 1) of the sensor element 101. The thermal shock resistant protective layer is provided to prevent cracking of the sensor element 101 due to thermal shock caused by moisture contained in the measurement gas adhering to the sensor element 101 and condensing when the gas sensor 100 is in use and to prevent poisoning substances coexisting in the measurement gas from entering into the sensor element 101. A laminar gap (gap layer) may be formed between the sensor element 101 and the thermal shock resistant protective layer.

The sensor element 101 is contained in an unillustrated containment member (casing) of metal so that a portion between a side of the gas inlet 10 and a side of the reference gas introduction space 43 is sealed to be airtight. The sensor element 101 and the containment member constitute a main body of the gas sensor 100. The main body is attached to a point of use, such as an engine exhaust pipe, when the gas sensor 100 is in practical use. Wires are drawn from the containment member in which electrical connection between the wires and each part of the sensor element 101 is secured, and are connected to the controller 110, various power supplies, and the like as appropriate.

### Operation in Normal Mode

When the gas sensor 100 having a configuration as described above measures the NOx concentration, the main pump cell 21 and, further, the auxiliary pump cell 50 are operated so that feedback control to make the oxygen concentration in the first internal space 20 and, further, the second internal space 40 constant is performed, and the measurement gas having a constant oxygen concentration is introduced into the third internal space 61, and reaches the measurement electrode 44. For example, when the measurement gas is a lean atmosphere, the measurement gas having oxygen partial pressure sufficiently reduced to a degree (e.g., 0.0001 ppm to 1 ppm) having substantially no effect on measurement of NOx is introduced into the third internal space 61.

The measurement electrode 44 reduces NOx in the reaching measurement gas to generate oxygen. While the oxygen is pumped out by the measurement pump cell 41, the NOx current Ip2 flowing at the pumping out has a constant functional relationship (hereinafter referred to as sensitivity characteristics) with the NOx concentration of the measurement gas.

The sensitivity characteristics are identified in advance prior to practical use of the gas sensor 100 using a plurality of types of model gases having known NOx concentrations, and data thereof is stored in the controller 110. In practical use of the gas sensor 100, a signal representing a value of the NOx current Ip2 flowing in accordance with the NOx concentration of the measurement gas is provided to the controller 110 on a moment-to-moment basis. The controller 110 successively calculates NOx concentrations based on the value and the identified sensitivity characteristics, and outputs values thereof as NOx sensor detection values. The NOx concentration of the measurement gas can thereby be grasped in almost real time using the gas sensor 100.

In the present embodiment, operation of the gas sensor 100 concerning identification of the NOx concentration as described above is referred to as operation of the gas sensor 100 in a normal mode.

While target values of the electromotive force V0, the electromotive force V1, and the electromotive force V2 in the main sensor cell 80, the auxiliary sensor cell 81, and the measurement sensor cell 82 when feedback control is performed on the main pump cell 21, the auxiliary pump cell 50, and the measurement pump cell 41 in the normal mode may be set as appropriate in accordance with a specific configuration and the size of each part of the sensor element 101, and, further, a usage condition, a usage pattern, and the like of the gas sensor 100, assume hereinafter that the target values of the electromotive force V0, the electromotive force V1, and the electromotive force V2 are respectively set to 250 mV, 385 mV, and 400 mV as an example. These values are values generally normally set when the oxygen-ion conductive solid electrolyte forming the sensor element 101 is zirconia.

### Operation in Element Protection Mode

It is assumed that the gas sensor 100 according to the present embodiment mainly operates in the above-mentioned normal mode, that is, identifies the NOx concentration of the measurement gas under a condition in which oxygen is relatively sufficiently contained in the measurement gas, such as the lean atmosphere.

More particularly, when the gas sensor 100 is operated in the normal mode, the main pump cell 21 operates so that the electromotive force V0 generated in the main sensor cell 80 has a predetermined value in accordance with a value desired as an oxygen concentration value (or an oxygen partial pressure value) in the first internal space 20, but the oxygen concentration of the measurement gas introduced into the first internal space 20 from the external space changes from moment to moment, so that the main pump cell 21 performs both pumping in and out of oxygen.

In contrast, while the auxiliary pump cell 50 and the measurement pump cell 41 can structurally pump in oxygen, values of the electromotive force V1 in the auxiliary sensor cell 81 and the electromotive force V2 in the measurement sensor cell 82 as control target values when these pump cells are operated are set based on the assumption that oxygen is pumped out in principle of measurement of the NOx concentration. That is to say, the auxiliary pump cell 50 and the measurement pump cell 41 exclusively pump out oxygen when the gas sensor 100 is operated in the normal mode.

The gas sensor 100, however, is not always used under an atmosphere in which oxygen is sufficiently contained, and is sometimes used in an environment in which an atmosphere gas can be a rich gas having a small air-fuel ratio, for example, when the main body of the gas sensor 100 is attached to an exhaust path of a gasoline engine, and the exhaust gas from the engine is the measurement gas. In this case, the measurement gas introduced into the sensor element 101 is the rich gas. In this case, the main pump cell 21 tries to maintain the oxygen concentration value in the first internal space 20 by pumping in oxygen from an outside.

When the measurement gas introduced into the sensor element 101 is extremely rich, however, an oxygen concentration uncontrollable condition in which a target oxygen concentration is not achieved in the first internal space 20 might occur as oxygen in an amount in accordance with the main pump voltage Vp0 applied to the main pump cell 21 cannot be pumped in from the outside even if the main pump voltage Vp0 is increased. In addition, the measurement electrode 44 disposed in the farthest portion of the gas distribution part can disadvantageously be over-reduced by the rich gas.

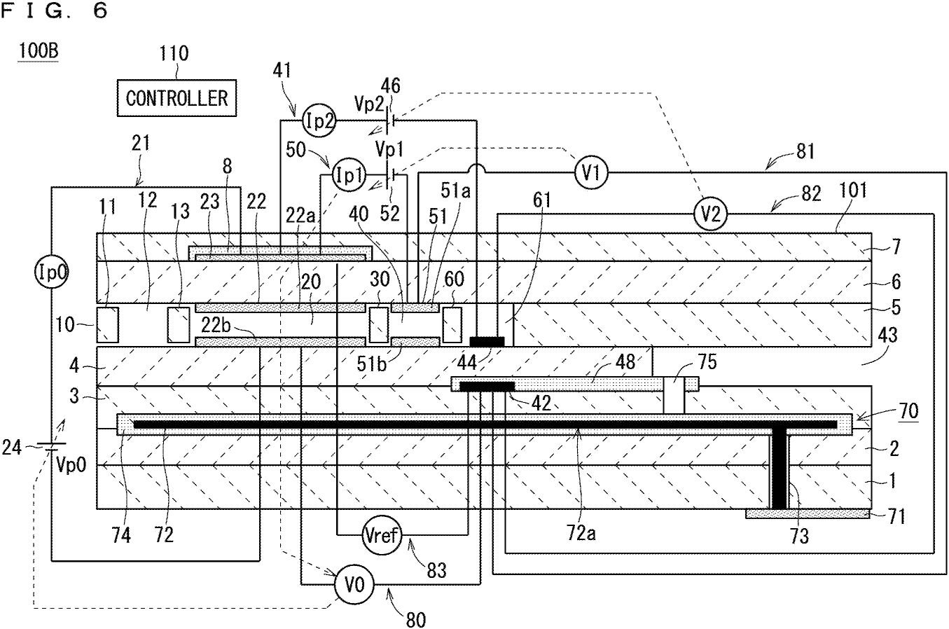

Such a condition in which oxygen cannot suitably be pumped in from the outside is likely to occur when movement in and out of the atmosphere gas around the outer pump electrode 23 where oxygen is taken from the external space is controlled by predetermined diffusion resistance as in the gas sensor disclosed in Japanese Patent Application Laid-Open No. 2021-162465. FIG. 6 is a diagram schematically showing one example of a configuration of a gas sensor 100B as one aspect of the gas sensor 100 having such a configuration. The gas sensor 100B has the same configuration as the gas sensor 100 illustrated in FIG. 1 except that the gas sensor 100B further includes a ceramic layer 7 and a porous body region 8 over the second solid electrolyte layer 6. The porous body region 8 is formed of a porous body (e.g., alumina) having a porosity of approximately 30% to 60% to cover the outer pump electrode 23 and to be exposed in opposite end portions in an unillustrated transverse direction of the element. The ceramic layer 7 is formed of ceramics (e.g., zirconia and alumina) that is dense to the same degree as the second solid electrolyte layer 6 and the like to cover the entire upper surface of the second solid electrolyte layer 6 including the porous body region 8. In the gas sensor 100B, movement in and out of the atmosphere gas around the outer pump electrode 23 is controlled by diffusion resistance provided by the porous body region 8.

In light of the foregoing, the gas sensor 100 according to the present embodiment can be operated in an element protection mode in which over-reduction of the measurement electrode 44 is avoided, and the measurement electrode 44 and, further, the sensor element 101 are preferentially protected while the oxygen concentration uncontrollable condition is avoided when the measurement gas is extremely rich.

The element protection mode has three aspects differing in procedures. These aspects will sequentially be described below.

### (First Aspect)

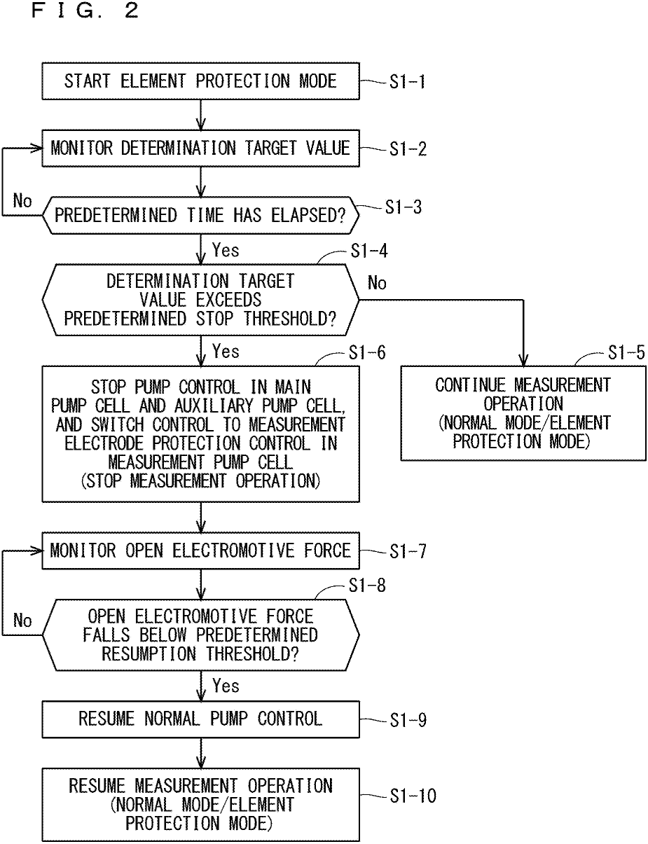

FIG. 2 is a diagram showing an operational flow in a first aspect of the element protection mode. The first aspect is generally a scheme of temporarily stopping NOx measurement operation of the gas sensor 100 including operation of pumping in oxygen from the outside to avoid over-reduction of the measurement electrode when the measurement gas is an extremely rich atmosphere.

In this aspect, the gas sensor 100 is first set to start operation in the element protection mode (step S1-1). This is achieved, for example, by a user (an operator) of the gas sensor providing appropriate setting instructions to the controller 110 through an unillustrated predetermined interface. Alternatively, the gas sensor 100 may be set to be always operated in the element protection mode.

Even after the start of the element protection mode, the NOx concentration is basically continuously measured as in the normal mode under control performed by the controller 110. An operation mode when the NOx concentration is measured as in the normal mode during execution of the element protection mode as described above is also particularly referred to as a basic mode. Operation in the normal mode is sometimes also referred to as operation in the basic mode. When the element protection mode is started, however, the controller 110 starts monitoring a predetermined determination target value in parallel with operation in the normal mode (step S1-2).

In this aspect, the determination target value is a value to be an indicator when whether to stop NOx measurement operation is determined. Specifically, an actual value of the electromotive force V0 is used. When the oxygen concentration of the measurement gas introduced into the first internal space 20 is lower than a target oxygen concentration in the first internal space 20 defined in advance, the main pump cell 21 pumps in oxygen to maintain the electromotive force V0 at a target value, but, when an extremely rich measurement gas is introduced, oxygen cannot sufficiently be pumped in, and the actual value of the electromotive force V0 deviates from the target value (becomes higher than the target value). A maximum value of the electromotive force V0 within which the deviation is allowed is set in advance as a stop threshold. The stop threshold is set to 350 mV, for example.

Monitoring of the determination target value is continued until the elapse (end) of a predetermined time (determination time) set in advance (step S1-3). The determination time is set to approximately 10 seconds, for example.

Upon the elapse (end) of the determination time (Yes in step S1-3), the controller 110 determines whether the determination target value exceeded the predetermined stop threshold during the determination time (step S1-4). Alternatively, whether the determination target value at the end of the determination time exceeds the predetermined stop threshold may be determined.

When the determination target value does not exceed the predetermined stop threshold during the determination time (No in step S1-4), the controller 110 continues measurement operation of the gas sensor 100 (step S1-5). The measurement operation may be continued in the normal mode by ending the element protection mode, or may be continued in the basic mode by starting the element protection mode again.

On the other hand, when there is a case that the determination target value exceeds the predetermined stop threshold during the determination time (Yes in step S1-4), the controller 110 transitions to a protected execution mode in which NOx measurement operation is stopped. Specifically, pump control (feedback control) in the main pump cell 21 and the auxiliary pump cell 50 is stopped, and, operation of the measurement pump cell 41 is switched to operation under measurement electrode protection control (step S1-6). Measurement electrode protection control is herein a control mode of operating the measurement pump cell 41 so that oxygen is pumped in to the third internal space 61 from the outside to protect the measurement electrode 44 against over-reduction by the extremely rich measurement gas. However, the absolute value of a pump-in voltage (the measurement pump voltage) Vp2 applied to the measurement pump cell 41 during measurement electrode protection control is set to 2000 mV or less to avoid cracking of the sensor element 101.

In this case, the main pump cell 21 and the auxiliary pump cell 50 are not operated, and thus the measurement gas introduced from the gas inlet 10 enters from the first internal space 20 to the third internal space 61 through the second internal space 40 as it is, whereas oxygen is pumped in to the third internal space 61 by the measurement pump cell 41, so that over-reduction of the measurement electrode 44 by the extremely rich measurement gas is suitably suppressed.

When pump control in the main pump cell 21 and the auxiliary pump cell 50 is stopped, actual values of the electromotive force V0 and the electromotive force V1 in the main sensor cell 80 and the auxiliary sensor cell 81 having been controlled to be predetermined constant values in accordance with a desired oxygen concentration so far become values in accordance with an unadjusted oxygen concentration of the measurement gas flowing into the first internal space 20 and the second internal space 40. Electromotive force generated in each sensor cell with pump control being stopped is particularly referred to as OPEN electromotive force.

The OPEN electromotive force varies in accordance with the oxygen concentration of the measurement gas flowing into each internal space, and has a value increasing with increasing oxygen concentration. The OPEN electromotive force can thus be used as an indicator of the magnitude of the oxygen concentration of the measurement gas present in the internal space corresponding to each sensor cell with pump control being stopped.

The controller 110 starts monitoring the OPEN electromotive force while stopping pump control in the main pump cell 21 and the auxiliary pump cell 50 and starting measurement electrode protection control targeted for the measurement pump cell 41 (step S1-7), and determines whether the OPEN electromotive force falls below a predetermined resumption threshold (step S1-8). In this case, the OPEN electromotive force in any one sensor cell (e.g., the main sensor cell 80) other than the measurement sensor cell 82 may be monitored to determine a magnitude relationship with the resumption threshold. Monitoring of the OPEN electromotive force is continued while the OPEN electromotive force does not fall below the resumption threshold (No in step S1-8).

The resumption threshold is set to a value so that it can be determined that the oxygen concentration of the measurement gas flowing into each internal space has increased to a degree to which pumping in or out of oxygen in each pump cell can be performed without any problems, even if pump control in each pump cell is resumed at a timing when the OPEN electromotive force has the set value. The resumption threshold can be set based on a correspondence relationship (functional relationship) between an air-fuel ratio of the measurement gas and a value of the OPEN electromotive force experimentally identified in advance, for example.

For example, when the exhaust gas of the gasoline engine is the measurement gas, even if an extremely rich exhaust gas flows into the sensor element 101 as the measurement gas at a certain timing, the flow is normally not permanent, and the oxygen concentration of the exhaust gas recovers to a degree to which each pump cell is suitably operated after the elapse of some time.

When the OPEN electromotive force in the main sensor cell 80 is to be monitored, the resumption threshold is set to 450 mV in one preferred example. It has been found in advance that an atmosphere in the first internal space 20 is stoichiometric composition or lean composition when the OPEN electromotive force is equal to or lower than 450 mV.

When it is determined that the OPEN electromotive force falls below the predetermined resumption threshold (Yes in step S1-8), the controller 110 resumes pump control operation having been stopped so far (step S1-9). That is to say, measurement electrode protection control targeted for the measurement pump cell 41 is stopped, the target values of the electromotive force V0, the electromotive force V1, and the electromotive force V2 in the main sensor cell 80, the auxiliary sensor cell 81, and the measurement sensor cell 82 are set again so that the oxygen concentrations in the first internal space 20, the second internal space 40, and the third internal space 61 have desired values, and each pump cell is operated again to achieve the target values.

When feedback control based on the target values of the electromotive force V0, the electromotive force V1, and the electromotive force V2 is eventually enabled in each pump cell, NOx measurement operation is resumed (step S1-10). The measurement operation may be continued in the normal mode by ending the element protection mode, or may be continued in the basic mode by starting the element protection mode again.

In this aspect, the measurement pump cell 41 pumps in oxygen to the third internal space 61 under measurement electrode protection control to protect the measurement electrode 44 against over-reduction while NOx measurement operation is stopped to avoid the oxygen concentration uncontrollable condition, so that the measurement electrode 44 promptly returns to a state in which normal feedback control can be performed in resuming NOx measurement operation.

As described above, in this aspect, when an extremely rich gas is introduced into the sensor element 101 as the measurement gas, the measurement pump cell 41 pumps in oxygen to the third internal space 61 to suppress over-reduction of the measurement electrode 44 while operation of the main pump cell 21 and the auxiliary pump cell 50 is stopped to avoid the oxygen concentration uncontrollable condition. Thus, excessive oxygen pump-in operation can be prevented to protect the pump cell, and over-reduction of the measurement electrode 44 can suitably be suppressed even when the extremely rich measurement gas reaches the third internal space 61. In resuming measurement of NOx upon determination that the oxygen concentration of the introduced measurement gas has recovered to a degree to which the pump cell can pump in oxygen, use of the measurement electrode can promptly be enabled.

### (Second Aspect)

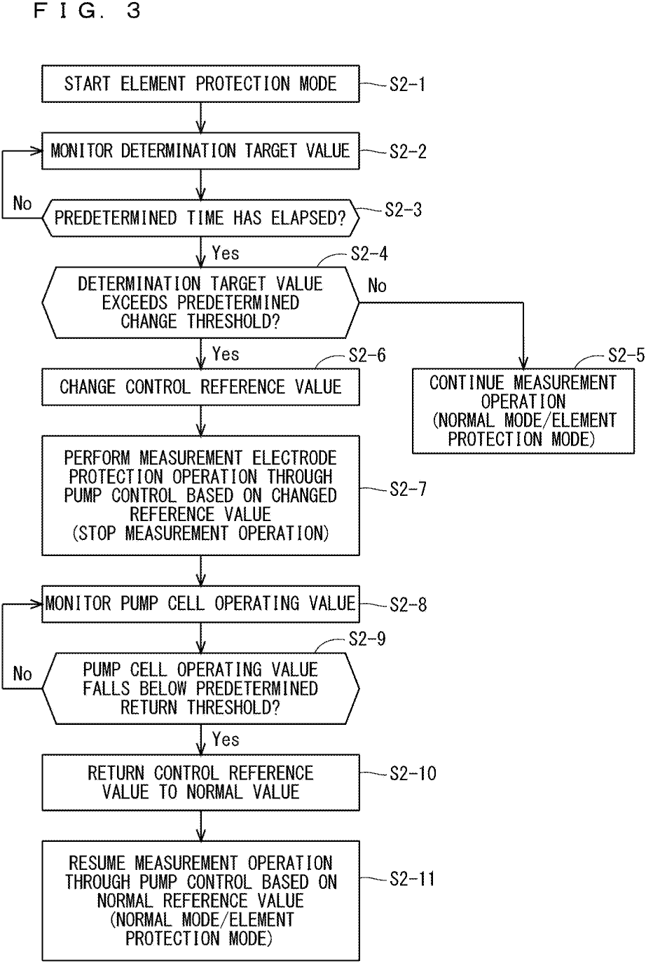

FIG. 3 is a diagram showing an operational flow in a second aspect of the element protection mode. The second aspect is generally a scheme of temporarily reducing the target oxygen concentration in the first internal space 20 to suppress excessive pumping in performed by the main pump cell 21 and to avoid over-reduction of the measurement electrode when the measurement gas is the extremely rich atmosphere.

Steps S2-1 to S2-5 in the second aspect are substantially similar to steps S1-1 to S1-5 in the first aspect. The determination target value is specifically the actual value of the electromotive force V0 as in the first aspect.

In this aspect, however, the determination target value is used as an indicator when whether to change the target oxygen concentration in the first internal space 20 is determined. More particularly, the determination target value is used as an indicator when a target value of the electromotive force V0 in the main sensor cell 80 in accordance with the target oxygen concentration is changed. The target value of the electromotive force V0 and the target oxygen concentration in the first internal space 20 have such a relationship that the target oxygen concentration in the first internal space 20 decreases with increasing target value of the electromotive force V0.

That is to say, in this aspect, a maximum value of the electromotive force V0 within which the deviation is allowed when the actual value of the electromotive force V0 deviates from the target value as a result of introduction of the extremely rich measurement gas into the first internal space 20 is set in advance as a change threshold. The controller 110 starts the element protection mode (basic mode) (step S2-1), starts monitoring the determination target value (step S2-2), and, upon the elapse (end) of a determination time (Yes in step S2-3), determines whether the determination target value exceeded the predetermined change threshold during the determination time (step S2-4) as in the first aspect. The change threshold may be the same as or may be different from the stop threshold in the first aspect.

When the determination target value does not exceed the predetermined change threshold during the determination time (No in step S2-4), the controller 110 continues the measurement operation of the gas sensor 100 in the normal mode or in the element protection mode (basic mode) (step S2-5) as in the first aspect.

On the other hand, when there is a case that the determination target value exceeds the predetermined change threshold during the determination time (Yes in step S2-4), the basic mode transitions to the protected execution mode also in this aspect. In this aspect, however, the controller 110 changes a control reference value in pump control performed in measuring the NOx concentration from a value (normal value) in the basic mode (normal mode) (step S2-6).

Specifically, the controller 110 at least changes the target value of the electromotive force V0 in the main sensor cell 80 to be a reference in the pump control in the main pump cell 21 to a value greater than the normal value (further, than the change threshold). For example, the target value of the electromotive force V0 is changed from 250 mV, which is the value in the normal mode, to 350 mV. The target value of the electromotive force V0 after the change is referred to as a changed reference value. The target value of the electromotive force V0 is changed in this manner, so that the target oxygen concentration in the first internal space 20 is reduced. In addition, a control reference value when pump control is performed in the auxiliary pump cell 50 may be changed from a value in the normal mode.

The controller 110 switches operation of the measurement pump cell 41 to operation under measurement electrode protection control while starting control based on the changed reference value for the main pump cell 21 (and, further, the auxiliary pump cell 50) (step S2-7). As a result, measurement of NOx is stopped in the protected execution mode also in this aspect. The absolute value of the pump-in voltage (measurement pump voltage) Vp2 applied to the measurement pump cell 41 during measurement electrode protection control is set to 2000 mV or less also in this aspect.

The target value of the electromotive force V0 is changed to the changed reference value greater than the normal value, and the target oxygen concentration in the first internal space 20 is reduced, so that a difference between the target oxygen concentration and the oxygen concentration of the extremely rich measurement gas entering into the first internal space 20 is smaller than that in the normal mode. When each pump cell is controlled based on the changed reference value, the amount of oxygen required to be pumped in to the first internal space 20 by the main pump cell 21 to achieve the target oxygen concentration is reduced compared with that before the change. As a result, an excessive increase in main pump current Ip0 or main pump voltage Vp0 in the main pump cell 21 is suppressed.

Furthermore, while the main pump cell 21 and the auxiliary pump cell 50 are stopped when the determination target value exceeds the stop threshold for transition to the protected execution mode in the first aspect, pump control operation in these pump cells itself is continued in this aspect, so that the time required from the end of the protected execution mode to resumption of measurement in this aspect is shorter than that in the first aspect.

The controller 110 causes the measurement pump cell 41 to start operation based on measurement electrode protection control and starts monitoring a pump cell operating value (step S2-8), and determines whether the pump cell operating value falls below a return threshold (step S2-9).

The pump cell operating value is specifically the main pump current Ip0 or the main pump voltage Vp0. The pump cell operating value increases with increasing amount of pumped in oxygen. The return threshold is set to a value so that it can be determined that the oxygen concentration of the measurement gas flowing into each internal space has increased to a degree to which pumping in or out of oxygen in each pump cell can be performed without any problems, even if the control reference value is returned from the changed reference value to the normal value at a timing when the pump cell operating value becomes the set value.

Monitoring of the pump cell operating value is continued unless the pump cell operating value falls below the return threshold (No in step S2-9).

On the other hand, when it is determined that the pump cell operating value falls below the predetermined return threshold (Yes in step S2-9), the controller 110 returns the control reference value from the changed reference value to the normal value (step S2-10). Then, operation of the measurement pump cell 41 based on measurement electrode protection control is stopped, and NOx measurement operation based on the normal control reference value is resumed instead (step S2-11). The measurement operation may be continued in the normal mode by ending the element protection mode, or may be continued by starting the element protection mode again.

As described above, in this aspect, when the extremely rich gas is introduced into the sensor element 101 as the measurement gas, the measurement pump cell 41 pumps in oxygen to the third internal space 61 to suppress over-reduction of the measurement electrode 44 while excessive pumping in performed by the main pump cell 21 is suppressed by temporarily reducing the target oxygen concentration in the first internal space 20 to avoid the oxygen concentration uncontrollable condition. Over-reduction of the measurement electrode 44 is thus suitably suppressed even when the extremely rich measurement gas reaches the third internal space 61 as in the first aspect. In contrast to the first aspect, operation of the main pump cell 21 and the auxiliary pump cell 50 is not stopped, so that use of the measurement electrode can promptly be enabled in resuming measurement of NOx upon determination that the oxygen concentration of the introduced measurement gas has recovered to a degree to which the pump cell can pump in oxygen.

### (Third Aspect)

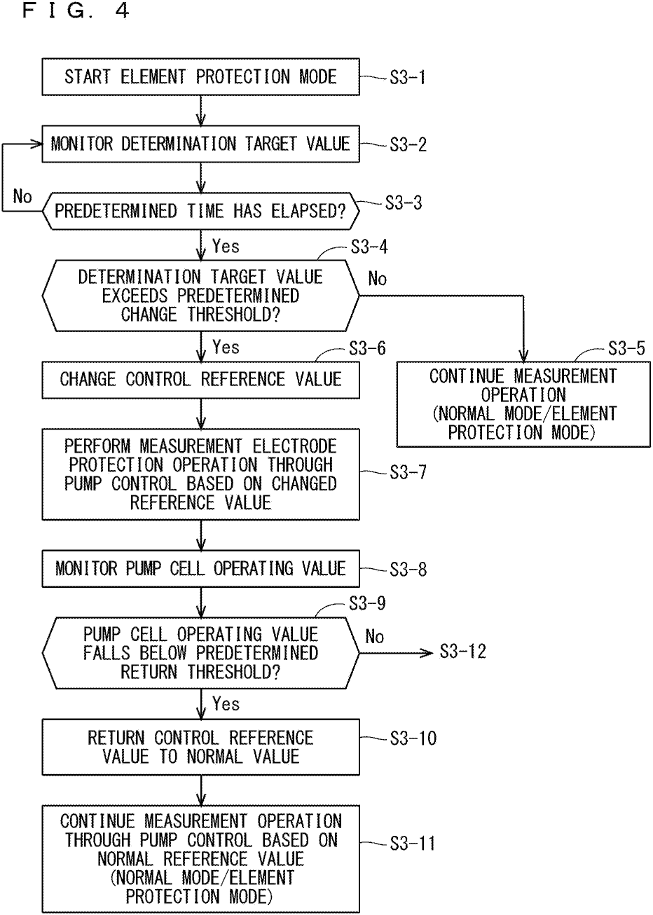

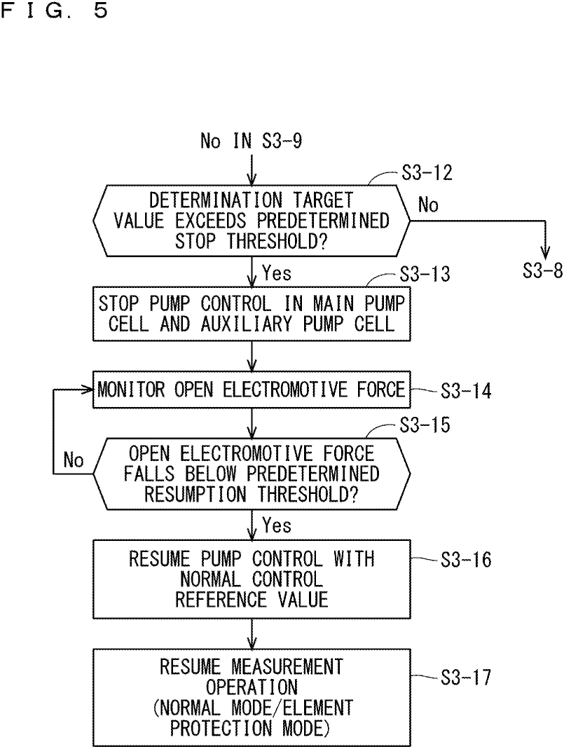

FIGS. 4 and 5 are diagrams showing an operational flow in a third aspect of the element protection mode. The third aspect is a scheme of making a response to the oxygen concentration uncontrollable condition in the main pump cell 21 in two stages in accordance with a degree thereof while avoiding over-reduction of the measurement electrode when the measurement gas is the extremely rich atmosphere by combining the first aspect and the second aspect.

In summary, when the measurement gas is the extremely rich atmosphere, excessive pumping in performed by the main pump cell 21 is first suppressed by temporarily reducing the target oxygen concentration in the first internal space 20 while measurement electrode protection control is performed for the measurement pump cell 41 as in the second aspect. When the oxygen concentration in the first internal space 20 is not sufficiently recovered, NOx measurement operation of the gas sensor 100 including operation of pumping in oxygen from the outside is temporarily stopped as in the first aspect.

Steps S3-1 to S3-7 in the third aspect are the same as steps S2-1 to S2-7 in the second aspect. That is to say, in the third aspect, the controller 110 first starts the element protection mode (basic mode) (step S3-1), starts monitoring the determination target value (step S3-2), and, upon the elapse (end) of the determination time (Yes in step S3-3), determines whether the determination target value exceeded the predetermined change threshold during the determination time (step S3-4) as in the second aspect. The determination target value is specifically the actual value of the electromotive force V0 also in this aspect.

When the determination target value does not exceed the predetermined change threshold during the determination time (No in step S3-4), the controller 110 continues the measurement operation of the gas sensor 100 in the normal mode or in the element protection mode (basic mode) (step S3-5) as in the first and second aspects.

On the other hand, when there is a case that the determination target value exceeds the predetermined change threshold during the determination time (Yes in step S3-4), the basic mode transitions to the protected execution mode also in this aspect. While the controller 110 changes the control reference value from the value (normal value) in the basic mode (normal mode) for the main pump cell 21 (and, further, the auxiliary pump cell 50) (step S3-6) to start control based on the changed reference value, operation of the measurement pump cell 41 is switched to operation under measurement electrode protection control (step S3-7). As a result, measurement of NOx is stopped. The absolute value of the pump-in voltage (measurement pump voltage) Vp2 applied to the measurement pump cell 41 during measurement electrode protection control is set to 2000 mV or less also in this aspect.

Monitoring of the pump cell operating value is also started (step S3-8), and, when it is determined that the pump cell operating value falls below the predetermined return threshold (Yes in step S3-9), the controller 110 returns the control reference value from the changed reference value to the normal value (step S3-10), stops operation of the measurement pump cell 41 based on measurement electrode protection control, and resumes NOx measurement operation based on the normal control reference value in the normal mode or in the element protection mode (basic mode) instead (step S3-11) as in the second aspect.

On the other hand, when it is determined that the pump cell operating value does not fall below the return threshold (No in step S3-9), whether the determination target value exceeds the predetermined stop threshold is determined (step S3-12). The stop threshold is set to a value greater than the change threshold in this aspect.

When the determination target value does not exceed the stop threshold (No in step S3-12), processing returns to step S3-8 to resume monitoring of the pump cell operating value, and processing in and after step S3-9 is performed again. A loop in which step S3-8, step S3-9, step S3-12, step S3-8, ... are performed is thus repeated in a condition in which the pump cell operating value is equal to or greater than the predetermined return threshold, but the determination target value is equal to or smaller than the stop threshold. This means that, when excessive pumping in of oxygen performed by the main pump cell 21 caused by introduction of the rich gas into the sensor element 101 can be avoided by reducing the target oxygen concentration as in the second aspect, handling in the aspect is continued. Measurement electrode protection control targeted for the measurement pump cell 41 is still continued also in this condition.

When the determination target value after the change of the control reference value exceeds the stop threshold (Yes in step S3-12), similar procedures to those in the first aspect are performed. The procedures are intended to achieve protection more surely when the pump cell cannot sufficiently be protected only by reducing the target oxygen concentration.

Specifically, the controller 110 first stops pump control in the main pump cell 21 and the auxiliary pump cell 50 (step S3-13). A pump cell operating in this state is thus only the measurement pump cell 41 for which measurement electrode protection control is performed. That is to say, when it is likely to be difficult to avoid excessive pumping in of oxygen performed by the main pump cell 21 by reducing the target oxygen concentration, operation of the main pump cell 21 and the auxiliary pump cell 50 is stopped to protect these pump cells.

The controller 110 further starts monitoring of the OPEN electromotive force in each sensor cell other than the measurement sensor cell 82 (step S3-14), and determines whether the OPEN electromotive force falls below the predetermined resumption threshold (step S3-15). When the OPEN electromotive force in the main sensor cell 80 is to be monitored, the resumption threshold is set to 450 mV in one preferred example. Monitoring of the OPEN electromotive force is continued while the OPEN electromotive force does not fall below the resumption threshold (No in step S3-15).

When it is determined that the OPEN electromotive force falls below the resumption threshold (Yes in step S3-15), the controller 110 stops measurement electrode protection control targeted for the measurement pump cell 41, then sets the control reference value to the normal value again for each pump cell, and resumes the pump control operation (step S3-16). When feedback control based on the electromotive force V0, the electromotive force V1, and the electromotive force V2 in each pump cell is eventually enabled, NOx measurement operation in the normal mode or in the element protection mode (basic mode) is resumed (step S3-17).

As described above, in this aspect, the first aspect and the second aspect are combined, and, while handling to avoid the oxygen concentration uncontrollable condition in the protected execution mode is switched in stages in accordance with richness of the measurement gas introduced into the sensor element 101, the measurement electrode 44 is protected against over-reduction as in the first and second aspects. Over-reduction of the measurement electrode 44 is thus suitably suppressed even when the extremely rich measurement gas reaches the third internal space 61. On the other hand, a condition in which the main pump cell 21 and the auxiliary pump cell 50 are stopped is minimized. In resuming measurement of NOx upon determination that the oxygen concentration of the introduced measurement gas has recovered to a degree to which the pump cell can pump in oxygen, use of the measurement electrode can promptly be enabled.

As described above, according to the present embodiment, the oxygen concentration uncontrollable condition due to difficulty in pumping in oxygen to the internal space is suitably avoided when the gas sensor is used in an environment in which the measurement gas can be the rich gas having a small air-fuel ratio, such as the exhaust gas from the gasoline engine. In addition, over-reduction of the measurement electrode by the rich gas is suitably suppressed, so that use of the measurement electrode can promptly be enabled in resuming measurement of NOx.

### Modifications

While the actual value of the electromotive force V0 is used as the determination target value in the above-mentioned embodiment, a value of the main pump current Ip0 when the main pump cell 21 pumps in oxygen may be used as the determination target value instead. The main pump current Ip0 can be an indicator of a degree of pumping in of oxygen to the first internal space 20 as it increases in accordance with amount of oxygen pumped in to the first internal space 20. The stop threshold in the first aspect and the change threshold in the second and third aspects are set in accordance with the determination target value to be used.

In the normal mode and the basic mode of the element protection mode, the gas sensor 100 may be operated through Ip1 constant control in which the auxiliary pump cell 50 is controlled so that the auxiliary pump current Ip1 having a constant magnitude flows, and, in this case, an actual value of the auxiliary pump current Ip1 may be used as the determination target value in the protected execution mode of the element protection mode in place of or in addition to the actual value of the electromotive force V0. The auxiliary pump current Ip1 when the auxiliary pump cell 50 pumps out oxygen is maintained at a constant value set in advance while the oxygen concentration of the measurement gas introduced from the first internal space 20 to the second internal space 40 is maintained at a predetermined value, so that the oxygen concentration in the second internal space 40 can be controlled at a predetermined value. When the measurement gas having a small oxygen concentration enters the second internal space 40 as a result of introduction of the extremely rich measurement gas into the first internal space 20 to disable adjustment of the oxygen concentration in the first internal space 20, however, the auxiliary pump cell 50 cannot pump out oxygen from the second internal space 40, and the actual value of the auxiliary pump current Ip1 decreases from the set constant value while the actual value of the electromotive force V1 increases from a constant value. The actual value of the auxiliary pump current Ip1 can thus also be used as the determination target value.

While the gas sensor includes the sensor element having three spaces therein in the above-mentioned embodiment, a configuration of the sensor element in which blackening can be caused by taking the extremely rich measurement gas into the sensor element is not limited to that in the above-mentioned embodiment.

Also in such a case, as long as the gas sensor includes the sensor element having the internal space in which the oxygen concentration is maintained constant by the electrochemical pump cell pumping in or out oxygen, the oxygen concentration uncontrollable condition of the sensor element can be avoided by applying the above-mentioned first to third aspects while modifying them as appropriate in accordance with a configuration of the element. Over-reduction of the measurement electrode by the rich gas can also suitably be suppressed. That is to say, the sensor element can suitably be protected under a rich gas atmosphere.

While the invention has been shown and described in detail, the foregoing description is in all aspects illustrative and not restrictive. It is therefore understood that numerous modifications and variations can be devised without departing from the scope of the invention.

What is claimed is:

1. A NOx sensor capable of sensing NOx in a measurement gas, the NOx sensor comprising:
a sensor element formed of an oxygen-ion conductive solid electrolyte; and
a controller controlling operation of the NOx sensor, wherein
the sensor element includes:
a plurality of internal spaces which communicate sequentially from an inlet for the measurement gas under predetermined diffusion resistance, and in which inner electrodes are respectively arranged;
an out-of-space pump electrode disposed at a location other than the plurality of internal spaces;
a reference electrode disposed to be contactable with a reference gas;
a plurality of electrochemical pump cells configured to pump in or out oxygen between the plurality of internal spaces and an outside of the sensor element by applying pump voltages across the respective inner electrodes and the out-of-space pump electrode from predetermined pump power supplies; and
a plurality of electrochemical sensor cells configured to cause potential differences between the respective inner electrodes and the reference electrode in accordance with oxygen concentrations in the respective internal spaces,
one of the inner electrodes arranged in the respective internal spaces is a measurement electrode capable of reducing NOx,
the plurality of electrochemical pump cells include:
a measurement pump cell including the measurement electrode; and
at least one oxygen concentration control pump cell including an inner electrode other than the measurement electrode,
the plurality of electrochemical sensor cells include:
a measurement sensor cell including the measurement electrode; and
at least one oxygen concentration sensing sensor cell including the inner electrode other than the measurement electrode, and
the controller
determines whether a predetermined determination target value exceeds a predetermined threshold during a predetermined determination time, the determination target value being an indicator of operation of pumping in oxygen of a determination target pump cell included in the at least one oxygen concentration control pump cell,
controls, unless the determination target value exceeds the predetermined threshold, the NOx sensor in a basic mode in which a concentration of NOx is identified based on a measurement pump current flowing between the measurement electrode and the out-of-space pump electrode of the measurement pump cell in accordance with the concentration of NOx due to reduction of NOx by the measurement electrode while an oxygen concentration in each of the plurality of internal spaces is maintained constant by operating each of the plurality of electrochemical pump cells, and
stops, when the determination target value exceeds the predetermined threshold, the basic mode, and controls the NOx sensor in a protected execution mode in which the measurement electrode is protected against over-reduction.

2. The NOx sensor according to claim 1, wherein
the predetermined threshold is a stop threshold to determine whether the at least one oxygen concentration control pump cell is required to be stopped, and
the controller
starts the protected execution mode when the determination target value exceeds the stop threshold, and, in the protected execution mode, operation of the at least one oxygen concentration control pump cell is stopped, the measurement pump cell is caused to pump in oxygen, and monitoring of a potential difference in the at least one oxygen concentration sensing sensor cell corresponding to the determination target pump cell is started, and
resumes control of the NOx sensor in the basic mode upon the potential difference subjected to monitoring falling below a predetermined resumption threshold.

3. The NOx sensor according to claim 1, wherein
the predetermined threshold is a change threshold to determine, when application of a pump voltage in the at least one oxygen concentration control pump cell is controlled, whether a target value of a potential difference in the at least one oxygen concentration sensing sensor cell corresponding to the at least one oxygen concentration control pump cell is required to be changed, and
the controller
starts the protected execution mode when the determination target value exceeds the change threshold, and, in the protected execution mode, control of the at least one oxygen concentration control pump cell is continued while the target value is changed to a value greater than a normal value, the measurement pump cell is caused to pump in oxygen, and monitoring of a pump cell operating value is started, the pump cell operating value being a value of the pump voltage or a value of a current in the at least one oxygen concentration control pump cell including at least the determination target pump cell, and
returns the target value to the normal value, and returns control of the NOx sensor to control in the basic mode upon the pump cell operating value falling below a return threshold.

4. The NOx sensor according to claim 1, wherein
the predetermined threshold is a change threshold to determine, when application of a pump voltage in the at least one oxygen concentration control pump cell is controlled, whether a target value of a potential difference in the at least one oxygen concentration sensing sensor cell corresponding to the at least one oxygen concentration control pump cell is to be changed, and
the controller
starts the protected execution mode when the determination target value exceeds the change threshold, and, in the protected execution mode, control of the at least one oxygen concentration control pump cell is continued while the target value is changed to a value greater than a normal value, the measurement pump cell is caused to pump in oxygen, and monitoring of a pump cell operating value is started, the pump cell operating value being a value of the pump voltage or a value of a current in the at least one oxygen concentration control pump cell including at least the determination target pump cell,
returns the target value to the normal value, and returns control of the NOx sensor to control in the basic mode upon the pump cell operating value falling below a return threshold,
continues monitoring of the pump cell operating value unless the pump cell operating value falls below the return threshold, and the determination target value exceeds a stop threshold greater than the change threshold,
starts monitoring of a potential difference in the at least one oxygen concentration sensing sensor cell corresponding to the determination target pump cell when the pump cell operating value does not fall below the return threshold, and the determination target value exceeds the stop threshold, and
resumes control of the NOx sensor in the basic mode upon the potential difference subjected to monitoring falling below a predetermined resumption threshold.

5. The NOx sensor according to claim 1, wherein
the determination target value is an actual value of a potential difference in the at least one oxygen concentration sensing sensor cell corresponding to the determination target pump cell.

6. The NOx sensor according to claim 1, wherein
the determination target value is a value of a pump current when the determination target pump cell pumps in oxygen.

7. The NOx sensor according to claim 1, wherein
the plurality of internal spaces include a first internal space, a second internal space, and a third internal space communicating sequentially via diffusion resistance parts,
the inner electrode other than the measurement electrode includes a main pump electrode disposed in the first internal space and an auxiliary pump electrode disposed in the second internal space,
the measurement electrode is disposed in the third internal space,
the at least one oxygen concentration control pump cell includes a main pump cell controlling an oxygen concentration in the first internal space and an auxiliary pump cell controlling an oxygen concentration in the second internal space,
the at least one oxygen concentration sensing sensor cell includes a main sensor cell configured to cause a potential difference between the main pump electrode and the reference electrode in accordance with an oxygen concentration in the first internal space and an auxiliary sensor cell configured to cause a potential difference between the auxiliary pump electrode and the reference electrode in accordance with an oxygen concentration in the second internal space, and
the controller identifies, at least in the basic mode, the concentration of NOx based on a magnitude of the measurement pump current flowing through the measurement pump cell in accordance with the concentration of NOx in the measurement gas having an adjusted oxygen concentration introduced into the third internal space while operating the main pump cell and the auxiliary pump cell so that the oxygen concentration in the first internal space and the oxygen concentration in the second internal space are each maintained at a predetermined constant value.

8. A NOx sensor operation method of operating a NOx sensor that includes a sensor element formed of an oxygen-ion conductive solid electrolyte, and is capable of sensing NOx in a measurement gas, wherein
the sensor element includes:
a plurality of internal spaces communicating sequentially under predetermined diffusion resistance from an inlet through which the measurement gas is introduced;
a plurality of inner electrodes arranged to face the respective internal spaces;
an out-of-space pump electrode disposed at a location other than the plurality of internal spaces;
a reference electrode disposed to be contactable with a reference gas;
a plurality of electrochemical pump cells configured to pump in or out oxygen between the plurality of internal spaces and an outside of the sensor element by applying pump voltages across the respective inner electrodes and the out-of-space pump electrode from predetermined pump power supplies; and
a plurality of electrochemical sensor cells configured to cause potential differences between the respective inner electrodes and the reference electrode in accordance with oxygen concentrations in the respective internal spaces,
one of the inner electrodes arranged in the respective internal spaces is a measurement electrode capable of reducing NOx,
the plurality of electrochemical pump cells include:
a measurement pump cell including the measurement electrode; and
at least one oxygen concentration control pump cell including an inner electrode other than the measurement electrode,
the plurality of electrochemical sensor cells include:
a measurement sensor cell including the measurement electrode; and
at least one oxygen concentration sensing sensor cell including the inner electrode other than the measurement electrode, and
the method comprises
a) determining whether a predetermined determination target value exceeds a predetermined threshold during a predetermined determination time, the determination target value being an indicator of operation of pumping in oxygen of a determination target pump cell included in the at least one oxygen concentration control pump cell,
unless the determination target value exceeds the predetermined threshold in the step a), the NOx sensor is controlled in a basic mode in which a concentration of NOx is identified based on a measurement pump current flowing between the measurement electrode and the out-of-space pump electrode of the measurement pump cell in accordance with the concentration of NOx due to reduction of NOx by the measurement electrode while an oxygen concentration in each of the plurality of internal spaces is maintained constant, and
when the determination target value exceeds the predetermined threshold in the step a), the basic mode is stopped, and the NOx sensor is operated in a protected execution mode in which the measurement electrode is protected against over-reduction.

9. The NOx sensor operation method according to claim 8, wherein
the predetermined threshold is a stop threshold to determine whether the at least one oxygen concentration control pump cell is required to be stopped,
when the determination target value exceeds the stop threshold in the step a), the protected execution mode is started,
in the protected execution mode,
operation of the at least one oxygen concentration control pump cell is stopped, the measurement pump cell is caused to pump in oxygen, and monitoring of a potential difference in the at least one oxygen concentration sensing sensor cell corresponding to the determination target pump cell is started, and
operation of the NOx sensor in the basic mode is resumed upon the potential difference subjected to monitoring falling below a predetermined resumption threshold.

10. The NOx sensor operation method according to claim 8, wherein
the predetermined threshold is a change threshold to determine, when application of a pump voltage in the at least one oxygen concentration control pump cell is controlled, whether a target value of a potential difference in the at least one oxygen concentration sensing sensor cell corresponding to the at least one oxygen concentration control pump cell is required to be changed,
when the determination target value exceeds the change threshold in the step a), the protected execution mode is started,
in the protected execution mode,
operation of the at least one oxygen concentration control pump cell is continued while the target value is changed to a value greater than a predetermined normal value, the measurement pump cell is caused to pump in oxygen, and monitoring of a pump cell operating value is started, the pump cell operating value being a value of the pump voltage or a value of a current in the at least one oxygen concentration control pump cell including at least the determination target pump cell, and
the target value is returned to the predetermined normal value, and operation of the NOx sensor is returned to operation in the basic mode upon the pump cell operating value falling below a return threshold.

11. The NOx sensor operation method according to claim 8, wherein
the predetermined threshold is a change threshold to determine, when application of a pump voltage in the at least one oxygen concentration control pump cell is controlled, whether a target value of a potential difference in the at least one oxygen concentration sensing sensor cell corresponding to the at least one oxygen concentration control pump cell is to be changed,
when the determination target value exceeds the change threshold in the step a), the protected execution mode is started,
in the protected execution mode,
operation of the at least one oxygen concentration control pump cell is continued while the target value is changed to a value greater than a normal value, the measurement pump cell is caused to pump in oxygen, and monitoring of a pump cell operating value is started, the pump cell operating value being a value of the pump voltage or a value of a current in the at least one oxygen concentration control pump cell including at least the determination target pump cell,
the target value is returned to the normal value, and control of the NOx sensor is returned to control in the basic mode upon the pump cell operating value falling below a return threshold,
monitoring of the pump cell operating value is continued unless the pump cell operating value falls below the return threshold, and the determination target value exceeds a stop threshold greater than the change threshold,
monitoring of a potential difference in the at least one oxygen concentration sensing sensor cell corresponding to the determination target pump cell is started when the pump cell operating value does not fall below the return threshold, and the determination target value exceeds the stop threshold, and
operation of the NOx sensor in the basic mode is resumed upon the potential difference subjected to monitoring falling below a predetermined resumption threshold.

12. The NOx sensor operation method according to claim 8, wherein
the determination target value is an actual value of a potential difference in the at least one oxygen concentration sensing sensor cell corresponding to the determination target pump cell.

13. The NOx sensor operation method according to claim 8, wherein
the determination target value is a value of a pump current when the determination target pump cell pumps in oxygen.

14. The NOx sensor according to claim 2, wherein
the determination target value is an actual value of the potential difference in the at least one oxygen concentration sensing sensor cell corresponding to the determination target pump cell.

15. The NOx sensor according to claim 3, wherein
the determination target value is an actual value of a potential difference in the at least one oxygen concentration sensing sensor cell corresponding to the determination target pump cell.

16. The NOx sensor according to claim 4, wherein
the determination target value is an actual value of the potential difference in the at least one oxygen concentration sensing sensor cell corresponding to the determination target pump cell.

17. The NOx sensor according to claim 2, wherein
the plurality of internal spaces include a first internal space, a second internal space, and a third internal space communicating sequentially via diffusion resistance parts,
the inner electrode other than the measurement electrode includes a main pump electrode disposed in the first internal space and an auxiliary pump electrode disposed in the second internal space,
the measurement electrode is disposed in the third internal space,
the at least one oxygen concentration control pump cell includes a main pump cell controlling an oxygen concentration in the first internal space and an auxiliary pump cell controlling an oxygen concentration in the second internal space,
the at least one oxygen concentration sensing sensor cell includes a main sensor cell configured to cause a potential difference between the main pump electrode and the reference electrode in accordance with the oxygen concentration in the first internal space and an auxiliary sensor cell configured to cause a potential difference between the auxiliary pump electrode and the reference electrode in accordance with the oxygen concentration in the second internal space, and
the controller identifies, at least in the basic mode, the concentration of NOx based on a magnitude of the measurement pump current flowing through the measurement pump cell in accordance with the concentration of NOx in the measurement gas having an adjusted oxygen concentration introduced into the third internal space while operating the main pump cell and the auxiliary pump cell so that the oxygen concentration in the first internal space and the oxygen concentration in the second internal space are each maintained at a predetermined constant value.

18. The NOx sensor according to claim 3, wherein
the plurality of internal spaces include a first internal space, a second internal space, and a third internal space communicating sequentially via diffusion resistance parts,
the inner electrode other than the measurement electrode includes a main pump electrode disposed in the first internal space and an auxiliary pump electrode disposed in the second internal space,
the measurement electrode is disposed in the third internal space,
the at least one oxygen concentration control pump cell includes a main pump cell controlling an oxygen concentration in the first internal space and an auxiliary pump cell controlling an oxygen concentration in the second internal space,
the at least one oxygen concentration sensing sensor cell includes a main sensor cell configured to cause a potential difference between the main pump electrode and the reference electrode in accordance with the oxygen concentration in the first internal space and an auxiliary sensor cell configured to cause a potential difference between the auxiliary pump electrode and the reference electrode in accordance with the oxygen concentration in the second internal space, and
the controller identifies, at least in the basic mode, the concentration of NOx based on a magnitude of the measurement pump current flowing through the measurement pump cell in accordance with the concentration of NOx in the measurement gas having an adjusted oxygen concentration introduced into the third internal space while operating the main pump cell and the auxiliary pump cell so that the oxygen concentration in the first internal space and the oxygen concentration in the second internal space are each maintained at a predetermined constant value.

19. The NOx sensor according to claim 4, wherein
the plurality of internal spaces include a first internal space, a second internal space, and a third internal space communicating sequentially via diffusion resistance parts,
the inner electrode other than the measurement electrode includes a main pump electrode disposed in the first internal space and an auxiliary pump electrode disposed in the second internal space,
the measurement electrode is disposed in the third internal space,
the at least one oxygen concentration control pump cell includes a main pump cell controlling an oxygen concentration in the first internal space and an auxiliary pump cell controlling an oxygen concentration in the second internal space,
the at least one oxygen concentration sensing sensor cell includes a main sensor cell configured to cause a potential difference between the main pump electrode and the reference electrode in accordance with the oxygen concentration in the first internal space and an auxiliary sensor cell configured to cause a potential difference between the auxiliary pump electrode and the reference electrode in accordance with the oxygen concentration in the second internal space, and
the controller identifies, at least in the basic mode, the concentration of NOx based on a magnitude of the measurement pump current flowing through the measurement pump cell in accordance with the concentration of NOx in the measurement gas having an adjusted oxygen concentration introduced into the third internal space while operating the main pump cell and the auxiliary pump cell so that the oxygen concentration in the first internal space and the oxygen concentration in the second internal space are each maintained at a predetermined constant value.

20. The NOx sensor according to claim 5, wherein
the plurality of internal spaces include a first internal space, a second internal space, and a third internal space communicating sequentially via diffusion resistance parts,
the inner electrode other than the measurement electrode includes a main pump electrode disposed in the first internal space and an auxiliary pump electrode disposed in the second internal space,
the measurement electrode is disposed in the third internal space,
the at least one oxygen concentration control pump cell includes a main pump cell controlling an oxygen concentration in the first internal space and an auxiliary pump cell controlling an oxygen concentration in the second internal space,
the at least one oxygen concentration sensing sensor cell includes a main sensor cell configured to cause a potential difference between the main pump electrode and the reference electrode in accordance with the oxygen concentration in the first internal space and an auxiliary sensor cell configured to cause a potential difference between the auxiliary pump electrode and the reference electrode in accordance with the oxygen concentration in the second internal space, and
the controller identifies, at least in the basic mode, the concentration of NOx based on a magnitude of the measurement pump current flowing through the measurement pump cell in accordance with the concentration of NOx in the measurement gas having an adjusted oxygen concentration introduced into the third internal space while operating the main pump cell and the auxiliary pump cell so that the oxygen concentration in the first internal space and the oxygen concentration in the second internal space are each maintained at a predetermined constant value.
